(19) United States
(12) Patent Application Publication
(10) Pub. No.: US 2021/0236247 A1
(43) Pub. Date: Aug. 5, 2021

(54) MEDICAL IMPLANT AND MEDICAL IMPLANT SYSTEM FOR MALAR PROCESS OF THE MAXILLA

(71) Applicant: João Manuel MENDES CARAMÊS, Lisboa (PT)

(72) Inventor: João Manuel MENDES CARAMÊS, Lisboa (PT)

(21) Appl. No.: 17/052,539

(22) PCT Filed: May 3, 2018
(86) PCT No.: PCT/PT2018/050016

§ 371 Date: Nov. 3, 2020

(51) Int. Cl. A61C 8/00; A61L 27/32

(52) U.S. Cl. CPC A61C 8/0034; A61L 27/32; A61C 8/0013; A61C 8/0089

(57) ABSTRACT

The invention consists of a medical implant for anchoring to the malar process of the maxilla, said implant having a head (1) with an angulation of 35° to 55°; an internal or external connector (2) for different types of abutments; an implant body (3) shaped to define an apical and basal portion, said apical portion being threaded all around, and said basal portion having threads on one side and a faceted canoe-thinning shape on the other side for better layering of soft tissues.

The invention also concerns a medical implant system comprising the aforementioned medical implant, a surgical guide and at least one bone drill.

The invention is suitable for rehabilitation of patients suffering from posterior edentulous maxillae with insufficient bone volume and allows for an immediate rehabilitation of the edentulous patient with a reduced treatment time and morbidity, through a considerably less invasive and technically simplified technique.

### FIELD OF THE INVENTION

The invention consists of a new surgical implant for the malar process of the maxilla, as well as a medical implant system with said medical implant, a surgical guide and at least one bone drill. The implant and system of the invention allow for an immediate rehabilitation of patients suffering from posterior edentulous maxillae with insufficient bone volume, with a reduced treatment time and morbidity, through a considerably less invasive and technically simplified technique, accessible to any generalist in implantology.

### PRIOR ART

Nowadays, dental implants are a widely used treatment method for partially and completely edentulous patients. The basis for modern dental implants is a biologic process called osseointegration, in which materials such as Titanium or Zirconia form an intimate bond with the bone. The implant fixture is first placed so that it is likely to osseointegrate, then a dental prosthetic such as a crown, bridge or denture is added.

Unfortunately, there are drawbacks in the use of oral implants. One is the lack of sufficient bone volume, particularly in the posterior maxilla. This insufficient bone volume can be due to bone resorption after teeth extraction, pneumatization of the sinus or a combination of both. Patients with moderate to severe atrophy challenge surgeons to discover alternative ways to use existing bone or resort to augmenting the patient with autogenous or alloplastic bone materials. A number of augmentation procedures have been suggested for these atrophied maxillae before implant placement, which include Le Fort I, onlay bone grafts and maxillary sinus graft procedures. These techniques reestablish adequate bone volume for implant placement, but they usually increase morbidity, are technically demanding and extend time until final rehabilitation as a wide range of healing times is required before dental implants can be placed and rehabilitated.

Developed by Prof. Branemark in 1988, Zygomatic implants have been documented as an alternative for patients showing severe atrophy of the maxilla. The main advantages of this technique are: avoiding graft and sinus lift procedures contributing to shorter and more comfortable treatment; reduced surgery time avoiding large grafting procedures; faster patient recovery and the simplification of the treatment of patients with severely atrophic maxillae. Further indications for zygomatic implants include failed conventional implant placement, failed sinus augmentation or grafting procedures, rehabilitation after tumor and trauma resections. Zygomatic implants described by Brånemark are self-tapping screws in commercially pure titanium with a well-defined machined surface. They are available in 8 different lengths, ranging from 30 to 52.5 mm. They present a unique 45° angulated head to compensate for the angulation between the zygoma and the maxilla. The portion that engages the zygoma, the apical two thirds, has a diameter of 4.0 mm and the portion that engages the residual maxillary alveolar process has a diameter of 4.5 mm to 5 mm.

In the classical protocol, zygomatic implants are inserted through the alveolar crest and maxillary sinus, using the zygomatic bone for anchorage. Usually one zygomatic implant is placed on each side of the atrophic maxilla, in combination with 2-4 conventional implants in the anterior region. Access to the maxillary sinus is necessary for visualization of the correct implant position. Preparation of access to the maxillary sinus is performed at a lateral posterior aspect in the future implant position and the Schneiderian membrane is elevated in an anterior direction. The implant is then placed and located at the inner aspect of the sinus wall, often without membrane perforation. Alternatively, the extrasinus placement approach has been described in order to reduce incidence of sinus complications and to improve the implant location and the emergence profile in a more crestal position. Due to the long drilling distance to the zygomatic bone and in order to protect critical adjacent anatomical structures, placement of zygomatic implants requires considerable surgical training and experience, as well as a meticulous diagnostic plan. To receive an adequate overview of the anatomical structures, presurgical 3D planning wit CT or CBCT scans is absolutely necessary.

Over the last two decades, zygomatic implants have proved to be an effective option in the management of the atrophic edentulous maxilla as well as for maxillectomy defects. Notwithstanding the significant advantages of zygomatic implants over other techniques, namely immediate restoration of the edentulous patient with immediate loading of the implants, shortened treatment period and avoiding grafting procedures, a very high level of surgical expertise is required due to the risk of compromising important anatomical structures. Severe complications have also been related with zygomatic implants in the literature.

Despite the reports of a high survival rate in zygomatic implants, multiple complications may arise with this technique, lowering the rate of success such as oronasal fistulas, Intraoral soft tissue problems, chronic sinusitis, orbital injury and intracranial penetration. To solve this problem, maximizing the advantages and minimizing the disadvantages, the implant system proposed for patent is a completely new approach to the rehabilitation of the resorbed posterior maxillae, using different anatomical areas, different implant designs and a specific surgical technique. This approach has the same objectives of traditional zygomatic implants, specifically allowing for immediate rehabilitation of the edentulous patient, reducing treatment time and morbidity but through a considerably less invasive, technically simplified technique, accessible to the generalist in implantology and above all, with a considerable lower probability of clinical risks and treatment failures.

### Solved Technical Problems

The implant and system of the invention allow for an immediate rehabilitation of patients suffering from posterior edentulous maxillae with insufficient bone volume, with a reduced treatment time and morbidity, through a considerably less invasive and technically simplified technique, accessible to any generalist in implantology.

This technology ultimately leads to safer procedures, with a considerable lower probability of clinical complications and treatment failures when compared to the techniques currently used for the edentulous atrophic maxilla (maxillary sinus graft procedures and zygomatic implants), and it aims to improve the quality of life of populations suffering from edentulism, making the process of recovering their fixed teeth through this technique easier, faster, more predictable and with less complications. One of the advantages of this technique is allowing the clinician to place teeth immediately on the day of the surgery following an immediate loading protocol. The patient can leave the clinic with the implant process complete allowing for a better quality of life.

### BRIEF DESCRIPTION OF THE DRAWINGS

FIG. 1 is an illustration of the Human skull as seen from the front and from below (with the jaw removed) highlighting the Malar Process of the maxilla (in black). Also known as the Zygomatic process of the maxilla (not to be confused with the zygomatic bone), the malar process is a rough triangular eminence situated at the angle of separation between the anterior, zygomatic, and orbital surfaces forming part of the anterior surface at the front. Behind it is concave and forms part of the infratemporal fossa. Above it is rough and serrated for articulation with the zygomatic bone. Below it presents a prominent arched border which marks the division between the anterior and infratemporal surfaces. The high quality and quantity of available bone in this area makes it ideal for implant stabilization.

FIG. 2 is a depiction of the general features of a malar implant containing a head (1) with an angulation of 35° to 55°; either internal or external connector (2) for different types of abutments; and an implant body (3) with a faceted surface for better layering of soft tissues.

FIG. 3 is a depiction of malar implants anchored in the malar process of the maxilla. The faceted surface of the implant body enables it to be leveled with the lateral wall of the maxilla.

FIG. 4 is a tissue breakaway view of a malar implant anchored in the malar process of the maxilla.

FIG. 5 is a general depiction of a malar implant guide that includes small holes (4) for fixing the malar guide to the maxillary wall with appropriate screws; sharp pitons (5) to help support the malar guide while screwing it, a channel (6) which guides the preparation drills and connector (7) for a handle.

FIG. 6 is a depiction of the malar implant guide placed on the lateral wall of the sinus, with the center curvature placed on the zygomatic arch.

FIG. 7 is a depiction of the malar implant guide in correct positioning, with the implantation hole already perforated.

FIG. 8 is a side view of the human skull, with the malar implant in place.

FIG. 9 Is a gross depiction of a malar implant drill, with a shank (8) with standard dimensions, a wider profile (9) that acts as a stopper when used in conjunction with the malar implant guide, a drill neck (10) with a round profile that slides on the malar guide and a body (11) with equal shape and length to that of the malar implant.

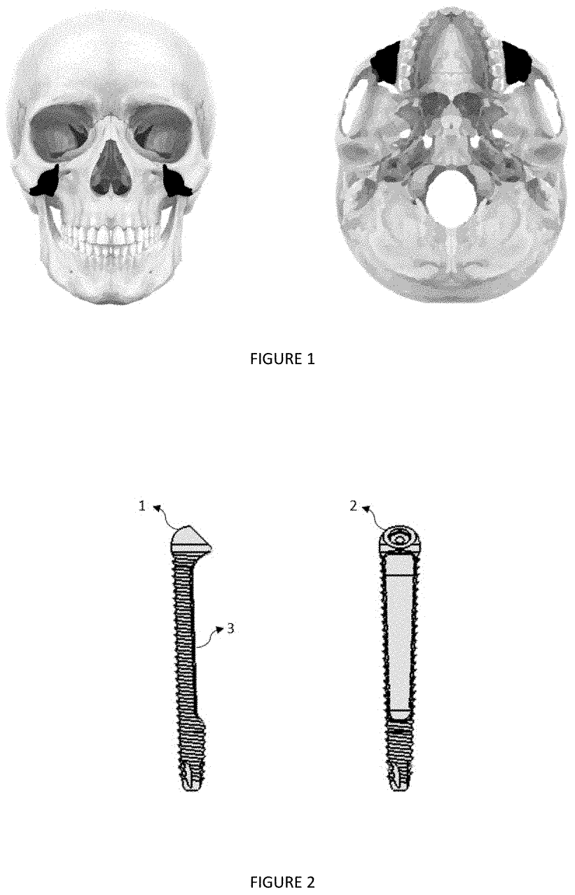

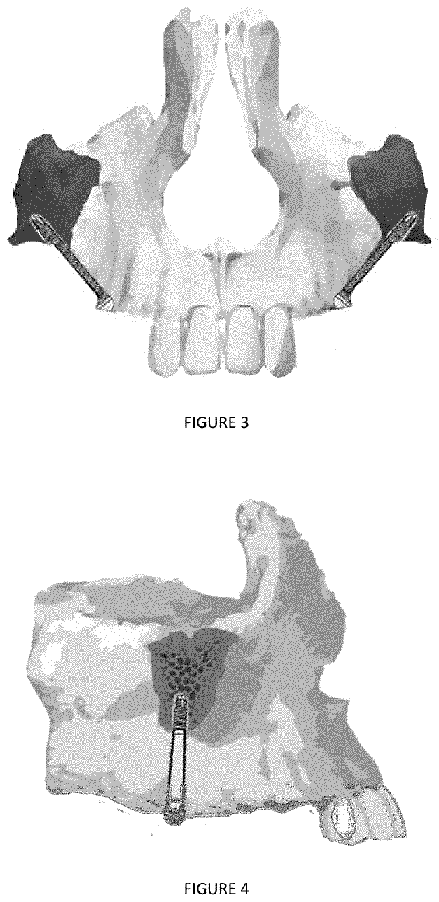

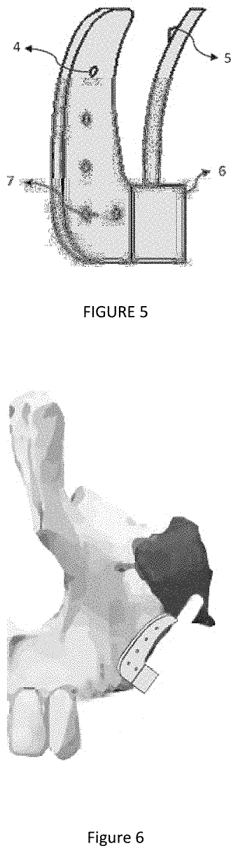

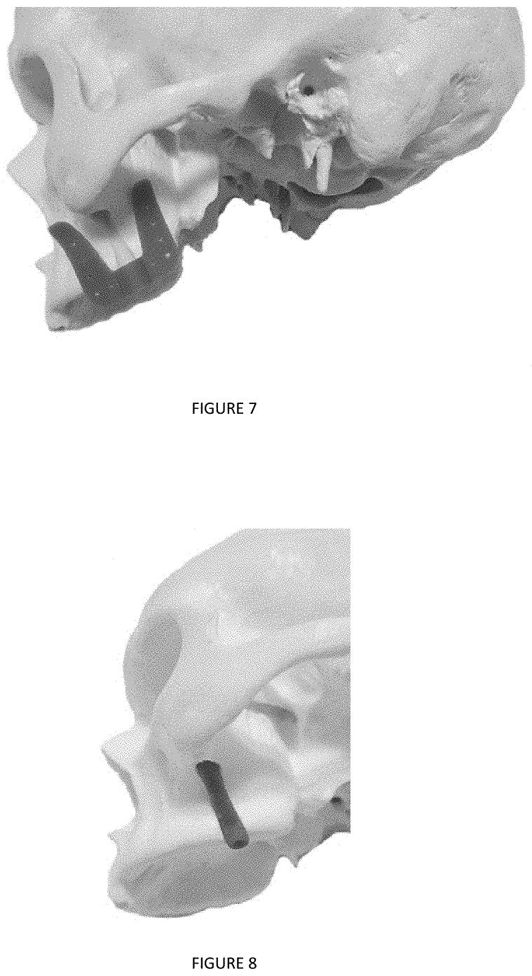

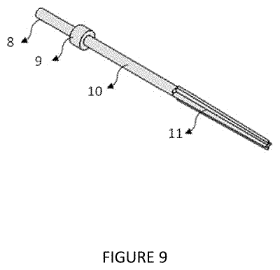

### DETAILED DESCRIPTION OF THE INVENTION

The present invention relates to a medical implant for anchoring to the malar process of the maxilla, said medical implant having a head (1) with an angulation of 35° to 55°; an either internal or external connector (2) for different types of abutments; and an implant body (3) which is shaped to define an apical and basal portion, said apical portion being threaded all around, and said basal portion having threads on one side and a faceted canoe-thinning shape on the other side for better layering of soft tissues.

In a preferred embodiment, the medical implant has a progressive diameter from 3 mm to 6 mm, and a length between 20 mm and 55 mm.

In another preferred embodiment, the medical implant is made of sintered titanium, machined titanium or zirconium.

In another preferred embodiment, the threads of the medical implant have rough surfaces, and the faceted surface of the body (3) and the head (1) of the implant have smooth surfaces.

In an even more preferred embodiment, the medical implant is made of titanium and the rough surfaces have a surface finish obtained by acid-etching, anodizing, medium blasting, laser ablation or plasma spraying to create adequate surface roughness; and the smooth surfaces have a surface finishing obtained by machining.

In another even more preferred embodiment, the medical implant is coated with an osteoconductive material, such as calcium phosphate, hydroxyapatite, calcium phosphosilicate or tricalcium phosphate.

In another even more preferred embodiment, the surface of the medical implant is doped with growth factors to improve early osseointegration, such as Bone Morphogenetic Proteins.

In another preferred embodiment, the medical implant is suitable for use in the rehabilitation of patients suffering from posterior edentulous maxillae with insufficient bone volume.

The present invention also relates to a medical implant system for surgical insertion in the malar process of the maxilla comprising:
a) the medical implant as described above;
b) a surgical guide;
c) at least one drill.

In a preferred embodiment, the surgical guide of the medical implant system is a plate with the configuration of the lateral wall of the sinus and arcuate towards the zygomatic arch, said plate having a flexible middle section to better adapt to different surface curvatures; a channel (6) which guides the preparation drills and fixation means.

In an even more preferred embodiment, the fixation means of the surgical guide is a connector (7) for a handle that may be inserted on either side of the guide.

In another even more preferred embodiment, the fixation means of the surgical guide comprise small holes (4) for fixing the surgical guide to the maxillary wall with appropriate screws and sharp pitons (5) to help supporting the surgical guide while screwing it.

In another even more preferred embodiment, the surgical guide is made of titanium or a polymeric material.

In another preferred embodiment, the at least one drill of the medical implant system consists of a pilot drill and a standard series of drills with increasing diameters, each drill having a shank (8) with a diameter of 3 to 6 mm; a wider profile (9) that acts as a stopper when used in conjunction with the surgical guide; a drill neck (10) with a round profile that slides on the surgical guide; and a body (11) with equal shape and length to that of the malar implant.

In another preferred embodiment, the medical implant system is suitable for use in the rehabilitation of patients suffering from posterior edentulous maxillae with insufficient bone volume.

Malar medical implants according to the invention are made of sintered titanium, machined titanium or zirconium, with progressive diameters of 3 to 6 mm and lengths comprised between 20 mm and 55 mm. The implant of the invention uses the malar/zygomatic process of the maxilla for anchorage instead of the zygomatic bone traditionally used. The lateral wall of the maxilla is hewn so as to partially support the device for better compressive and lateral stability. The apical part of the implant is threaded all around, and the body of the implant has threads on one side and a faceted canoe-thinning shape on the other for better layering of soft tissues. The implant head is angulated at between 35° and 55°, with either internal or external connections for different types of abutments.

Malar process medical implants may have one or a combination of different surface treatments to improve osseointegration and reduce the incidence of surgical site infection. Ideally, rough surfaces should be employed on the threads, while the faceted surface and implant head, in contact with soft tissues, should be smooth. On the titanium variant of the implant, the surface finish may be obtained using different techniques. Acid-etching is a common method, where strong acids create a rough surface on the implant. Anodizing, where an electrochemical process thickens and roughens the titanium oxide surface layer, also provides a good surface treatment for osseointegration. Moreover, medium blasting, laser ablation and plasma spraying may also be used to create adequate surface roughness. Conversely, machining may be utilized to obtain a smooth surface. Optionally, the implant may be coated with an osteoconductive material, such as calcium phosphate, hydroxyapatite, calcium phosphosilicate or tricalcium phosphate. The surface of the malar implants may also be doped with growth factors to improve early osseointegration, such as Bone Morphogenetic Proteins (BMP's).

The guide consists of a titanium plate with the configuration of the lateral wall of the sinus and arcuate towards the zygomatic arch, allowing for the direct visualization of the malar process of the maxilla. At the top of this device there is a channel which guides the implant site preparation drills. There are two methods of stabilization of the titanium plate: A handle can be adapted to connection in the plate. There are small pitons that stick to the maxillary bone wall. The titanium plate can be bent to better adapt to different surface curvatures. Since the plate is perforated, another possibility is to use small screws to attach it to the bone wall. In order to adapt the guide to different patient anatomies, three sizes of surgical guides were developed: number one, two and three.

A special set of drills was invented to be used in conjunction with the malar surgical guide. The set includes a pilot drill and a standard series of drills with increasing diameters. The flute length of the standard drills equals that of each implant. The neck is designed in the form of a cylinder to fit into the guide plate channel, with appropriate length for each implant size, limiting the maximum perforation length. The shank length and diameter are standard.

### Implantation Technique

The disclosed technique is a completely original approach to the rehabilitation of the resorbed posterior edentulous maxillae. The technique utilizes a completely different anatomical area—the malar process of the maxillary bone—instead of the zygomatic bone. It allows for an immediate rehabilitation of the edentulous patient with a reduced treatment time and morbidity, but through a considerably less invasive and technically simplified technique, accessible to any generalist in implantology. A novel malar process implant was specifically invented for this new technique, as well as a surgical guide and a bone drill intended to work in conjunction with the guide.

Pre-surgical planning focuses on the general health condition of the patient, the localized health condition of the mucous membranes and the jaws and the shape, size, and position of the bones of the jaws and adjacent and opposing teeth. There are few health conditions that absolutely preclude placing implants, although there are certain conditions that can increase the risk of failure. Those with poor oral hygiene, heavy smokers and diabetics are all at greater risk of long-term failure. Long-term steroid use, osteoporosis and other diseases that affect the bones can increase the risk of early failure of the implants.

Due to the long drilling distance to the malar process and in order to protect critical adjacent anatomical structures, placement of the invented malar implants requires considerable surgical training and meticulous diagnostic planning. To receive an adequate overview over the anatomical structures, presurgical 3D planning with CT or CBCT scans is a precondition. Cone beam computed tomography (CBCT) is an essential tool for treatment planning and post-procedure monitoring. By providing highly accurate 3-D images of the patient's anatomy from a single, low-radiation scan, CBCT technology delivers a comprehensive understanding of the patient's jaw and the anatomical structures necessary to properly provide treatment.

To ensure a successful implant placement of this new technique, one must take into account the patient's prosthetic needs, functional requirements, and anatomical constraints. During the assessments, 3-D imaging contributes to a greater success rate due to its ability to visualize previously undetectable anatomic variability. A CBCT study of the malar process is mandatory for a successful treatment.

To be effective, preoperative medication should be administered timeously. However, because the described technique is simpler, faster and less invasive, the need for aggressive medication is greatly diminished.

This pre-medication protocol significantly reduces the probability of dental implant failure when placed in normal conditions. However, it is important to take into account the individual medical condition of each patient and adjust the pre-medication protocol accordingly.

Although current zygomatic implant protocols require general anesthesia with nasal intubation followed by local infiltrative anesthesia, the described technique used in conjunction with the invented product allow the patient to be operated on with only local anesthesia, making the procedure much simpler and safer.

The incision technique includes a midcrestal incision (slightly palatal) and vertical releasing incisions in the molar region along the posterior part of the infra zygomatic crest in the direction of the zygoma, avoiding damage to the stennon duct from the parotid gland. A mucoperiosteal flap is then raised unilaterally to expose the alveolar crest, the infraorbital nerve, and the lateral wall of the maxilla to the superior rim of the zygomatic arch. These incisions are made with a surgical scalpel number 15c attached to scalpel handle number 3. The retraction of the tissues should be done with care to avoid damage or compression of the infra-orbital nerve. Two anatomical landmarks should be assessed: the vertical ridge/anterior border of the zygomatic arch and the lateral orbital border. A mucoperiosteal flap should be elevated to expose the central/posterior part of the malar process of the maxilla.

The path of the implant body will vary from being totally intrasinus to being totally extra-sinus. In other words, the new approach proposed for the placement of the malar implant is neither ‘internal’ nor ‘external’ to the sinus wall but, instead, promotes the placement of the malar implant according to the anatomy of the patient. The preparation of the implant site is now guided by the anatomy of the area, and no initial window is opened at the lateral wall of the maxillary sinus.

Upon visual inspection, the ideal size of the surgical guide is decided and banded into the lateral bone wall of the maxilla. When in the correct place (with the channel pointing to the malar process), the titanium plate is stabilized by pressing the pitons against the bone wall. Although in most cases the pitons are sufficient to stabilize the guide, it is preferable to screw the plate to the bone wall to avoid movement while drilling.

Following proper soft tissue retraction, the osteotomy sequence is initiated with a pilot bur. A groove is prepared as the drill moves into the malar bone. When the drill begins to pierce the malar bone, resistance must be felt and a depth of approximately 9 mm is perforated. Following this, the implant site preparation continues with a standard series of drills with increasing diameters. The second bur widens the previously prepared implant site. In cases of hard bone, a third bur is used to widen the site in order to avoid excessive torque when placing the implant, which may over-compress the bone.

The invented self-tapping implant is placed with a mount, in a contra angle aided by a motor, or manually. Usually, final insertion of the implant is manually driven. Bending forces should not be applied during this procedure.

The implant is placed with the 9 mm apical part inside the high-quality malar bone, while the rest of the implant body is external and rests against the lateral wall of the maxilla in a slot carved into the bone. The coronal part is placed deep into the bone crest. Thus, this part of the implant is supported and well stabilized allowing for effective healing of the peri-implant tissues. The primary stability achieved can be measured with the Periotest or RFQ devices. After confirming the implant mount screw is not loose (retighten if needed), it should be unscrewed by a central screw.

There are two options for closing: In a one-stage technique, the final abutment should be placed, and a full arch immediate loading protocol followed. In a two-staged technique, the cover screw must be placed to seal the implant and the mucoperiosteal flap closed. A second intervention is required, 4 months after the healing period. After determining the implant position, a fenestration should be made, slightly palatal to the incision, and a flap retracted.

The prosthetic clinical procedures follow the same sequence as a conventional implant-supported rehabilitation. Horizontal simple interrupted sutures (reabsorbable 4/0 polyglactin 910 suture) should close the flap.

One of the advantages of this technique is allowing the clinician to place teeth immediately on the day of the surgery following an immediate loading protocol. The patient can leave the clinic with the implant process complete allowing for a better quality of life.

Sutures are removed 1 week after the surgery and a post-op follow-up evaluation must be performed at 1, 3 and 6 months, to prepare the stage two procedure if it this the case.

Postoperative medication depends on the medical evaluation of the individual patient. As the procedure is less aggressive than traditional techniques, the premedication suggested may be enough.

Four months after surgery, an imprint is made using appropriate components and a fixed screw retained prosthesis is produced. The definitive prosthesis is screwed using preformed abutments, tilted at an angle of 0° to 30°.

1. Medical implant for anchoring to the malar process of the maxilla characterized by a head (1) with an angulation of 35° to 55°; either an internal or external connector (2) for different types of abutments; and an implant body (3) which is shaped to define an apical and basal portion, said apical portion being threaded all around, and said basal portion having threads on one side and a faceted canoe-thinning shape on the other side for better layering of soft tissues.

2. Medical implant according to claim 1 characterized in that it has a progressive diameter from 3 mm to 6 mm, and a length between 20 mm and 55 mm.

3. Medical implant according to claim 1 characterized in that it is made of sintered titanium, machined titanium or zirconium.

4. Medical implant according claim 1 characterized in that the threads of said medical implant have rough surfaces, and the faceted surface of the body (3) and the head (1) of said medical implant have smooth surfaces.

5. Medical implant according to claim 16 characterized in that it is made of titanium; the rough surfaces have a surface finish obtained by acid-etching, anodizing, medium blasting, laser ablation or plasma spraying to create adequate surface roughness; and the smooth surfaces have a surface finishing obtained by machining.

6. Medical implant according to claim 1 characterized in that it is coated with an osteoconductive material, such as calcium phosphate, hydroxyapatite, calcium phosphosilicate or tricalcium phosphate.

7. Medical implant according to claim 4 characterized in that the surface is doped with growth factors to improve early osseointegration, such as Bone Morphogenetic Proteins.

8. Medical implant according to claim 1 for use in the rehabilitation of patients suffering from posterior edentulous maxillae with insufficient bone volume.

9. Medical implant system for surgical insertion in the malar process of the maxilla characterized by comprising:
a) the medical implant as described in claim 1;
b) a surgical guide;
c) at least one drill.

10. Medical implant system according to claim 9 characterized in that the surgical guide is a plate with the configuration of the lateral wall of the sinus and arcuate towards the zygomatic arch, said plate having a flexible middle section to better adapt to different surface curvatures; a channel (6) which guides the preparation drills and fixation means.

11. Medical implant system according to claim 10 characterized in that the fixation means of the surgical guide is a connector (7) for a handle that may be inserted on either side of the guide.

12. Medical implant system according to claim 9 characterized in that the fixation means of the surgical guide comprise small holes (4) for fixing the surgical guide to the maxillary wall with appropriate screws and sharp pitons (5) to help supporting the surgical guide while screwing it.

13. Medical implant system according to any of the claim 9 characterized in that the surgical guide is made of titanium or a polymeric material.

14. Medical implant system according to claim 9 characterized in that the at least one drill consists of a pilot drill and a standard series of drills with increasing diameters, each drill having a shank (8) with a diameter of 3 to 6 mm; a wider profile (9) that acts as a stopper when used in conjunction with the surgical guide; a drill neck (10) with a round profile that slides on the surgical guide; and a body (11) with equal shape and length to that of the malar implant.

15. Medical implant system according to claim 9 for use in the rehabilitation of patients suffering from posterior edentulous maxillae with insufficient bone volume.

16. Medical implant according to claim 1 characterized in that it has a progressive diameter from 3 mm to 6 mm, and a length between 20 mm and 55 mm and is made of sintered titanium, machined titanium or zirconium.

17. Medical implant according to claim 1 characterized in that it is made of titanium and have rough surfaces, the rough surfaces have a surface finish obtained by acid-etching, anodizing, medium blasting, laser ablation or plasma spraying to create adequate surface roughness; and the smooth surfaces have a surface finishing obtained by machining; the medical implant coated with an osteoconductive material, such as calcium phosphate, hydroxyapatite, calcium phosphosilicate or tricalcium phosphate.

18. Medical implant according to any of the claim 5 characterized in that it is made of titanium; the rough surfaces have a surface finish obtained by acid-etching, anodizing, medium blasting, laser ablation or plasma spraying to create adequate surface roughness; and the smooth surfaces have a surface finishing obtained by machining; the surface being doped with growth factors to improve early osseointegration, such as Bone Morphogenetic Proteins.
